(19) United States
(12) Patent Application Publication
(10) Pub. No.: US 2017/0020430 A1
(43) Pub. Date: Jan. 26, 2017

(54) DATA DRIVEN ASSESSMENT APPARATUS AND METHOD

(71) Applicant: SurePeople LLC, Arlington Heights, IL (US)

(72) Inventor: NIKO DRAKOULIS, ARLINGTON HEIGHTS, IL (US)

(21) Appl. No.: 15/090,214

(22) Filed: Apr. 4, 2016

Related U.S. Application Data
(60) Provisional application No. 62/142,780, filed on Apr. 3, 2015.

(51) Int. Cl. A61B 5/16; G09B 5/02; G09B 7/00

(52) U.S. Cl. CPC A61B 5/167; G09B 7/00; G09B 5/02

(57) ABSTRACT

A data driven assessment apparatus and method generates assessment data of an individual's traits. The apparatus and method use a cloud based approach to assessment analysis which provides business administrators and individuals with personality profiles.

CROSS REFERENCE TO CO-PENDING APPLICATION

This application claims priority benefit to the April 3, 2015 filing date of co-pending U.S. Provisional Patent Application Ser. No. 62/142,780 filed in the name Niko Drakoulis as inventor of a “Data Driven Talent Management and Talent Acquisition Apparatus and Method”, the entire contents of which are incorporated herein in its entirety.

### BACKGROUND

The present data driven assessment apparatus and method relates, in general, to trait assessment tools.

Successful businesses require a motivated team of employees and administrators working toward a common business goal. However, every person has different traits, such as motivation, intelligence, personality and personality under pressure decision making and conflict management which make team interaction and achievement of business goals a hit or miss prospect at best.

### SUMMARY

A data driven assessment apparatus and method for generating on an individual's assessment data of the individual's traits integrated.

In one aspect, the method for generating an individual's at least one assessment data include accessing a memory by a computer processor coupled through a network to individual processors; administering an assessment through at least one the individual processor to determine an individual's assessment data, analyzing the assessment data by the computer processor, and generating an assessment report to the at least one individual processor containing at least one assessment of the traits of the individual.

In another aspect, the method includes storing an assessment test in a medium accessible by the computer processor and the step of administering an assessment includes administering an assessment test through the network to the at least one individual processor via a visual display.

In another aspect, the method includes administering an assessment to an individual in response to a request for the individual to take an assessment test by a third party request through at least one individual processor to the computer processor.

In another aspect, the method includes transmitting an assessment report from the computer processor to at least one of the individual processor of the individual and to another individual processor of the third party.

An apparatus for generating an individual's assessment data includes a computer processor assessing a memory storing an assessment test at least one individual processor coupled through a network to the computer processor, and a digital display coupled to the at least one individual processor for displaying an assessment test and an assessment report.

The apparatus and method measures an individual's emotional intelligence, relational intelligence and team intelligence by analyzing multiple unique traits based on a plurality of attributes. Assessing these attributes in a company's employees enables a business administrator to better understand the company's employees' personalities, strengths, growth opportunities and capacities.

### BRIEF DESCRIPTION OF THE DRAWING

The various features, advantages and other uses of the present apparatus and method will become more apparent by referring to the following detailed description and drawing.

FIG. 1 is a block diagram of the components of the data driven assessment apparatus.

FIGS. 2A and 2B show a flow diagram of the business administrator login procedure for the data driven assessment method.

FIGS. 3A and 3B show a flow diagram of the business dashboard.

FIGS. 4A and 4B show a flow diagram of a business request for PRISM LENS™ assessment.

FIG. 5 is a flow diagram of a business sequence to take a PRISM LENS™ assessment.

FIG. 6 is a flow diagram of an individual sign up procedure for the data driven assessment apparatus and method.

FIGS. 7A and 7B show a flow diagram of the individual login sequence.

FIGS. 8A and 8B show a flow diagram of the individual dashboard of the present apparatus and method.

FIGS. 9A and 9B show a flow diagram of an individual request for a PRISM LENS™ assessment.

FIG. 10 is a flow diagram of the sequence for an individual to take a PRISM LENS™ assessment.

FIGS. 11A-11I are pictorial representations of sample assessment questions.

FIGS. 12A-12D show a pictorial representation of an assessment report of an individual's primary personality.

FIGS. 13A-13D show a pictorial representation of personality under pressure assessment results of an individual.

FIGS. 14A-14C show a pictorial representation of processing blueprint assessment results of an individual.

FIGS. 15A-15C show a pictorial representation of motivation assessment results of an individual.

FIGS. 16A and 16B show a pictorial representation of conflict management assessment results of an individual.

FIG. 17 is a pictorial representation of fundamental need assessment results of an individual.

FIGS. 18A and 18B show a pictorial representation of decision making assessment results of an individual.

FIGS. 19A and 19B show a pictorial representation of primary learning style assessment results of an individual.

FIGS. 20A-20C show a pictorial representation of the combined assessment results depicted in FIGS. 12A-19B.

### DETAILED DESCRIPTION

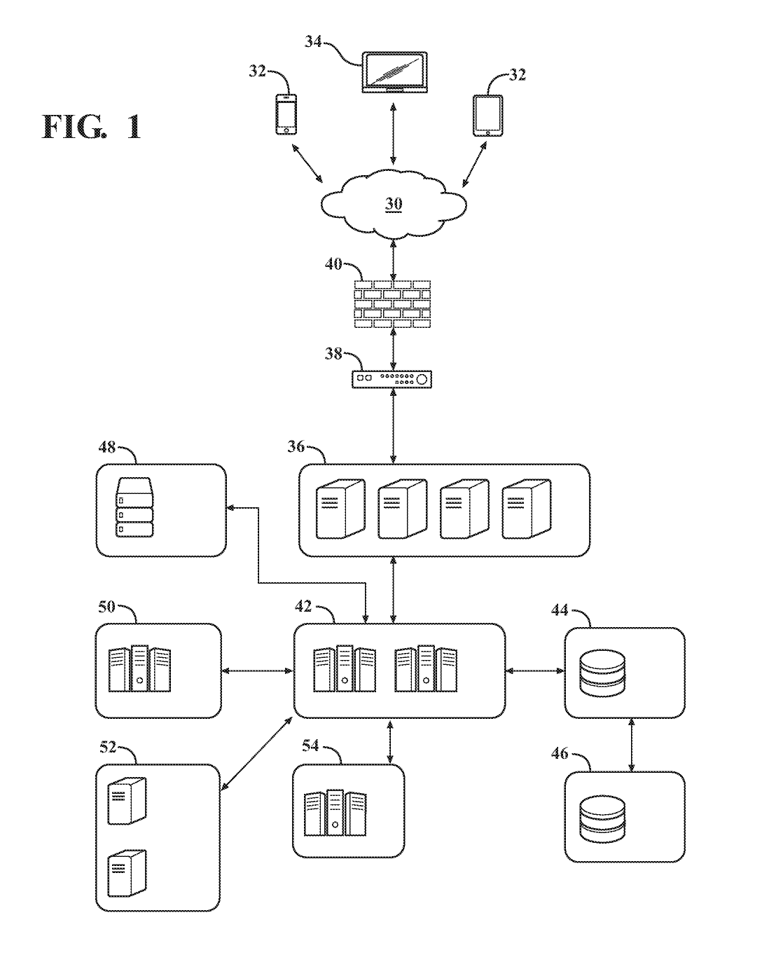

FIG. 1 depicts a cloud-based apparatus using a network, such as the worldwide web 30, for interaction between employers and employee via smartphones 32 and computers 34 to acquire employee assessment information, as well as reporting the results of such information to business management or to the individual.

As shown in FIG. 1, the apparatus, by example, includes a computer processor, which can be in the form of one or more web servers 36 connected to the worldwide web 30 through a load balancer 38 and a firewall 40. The web servers 36 communicate with one or more application servers 42. The application servers 42 communicate with database servers 44 and backup database servers 46, as well as data storage servers 48, a payment gateway server 50, a third party content services server 52 and CMS servers 54 (central management servers).

The present apparatus and method provide assessment-related reports to both business management and employees. The business side of the apparatus and method will first be described.

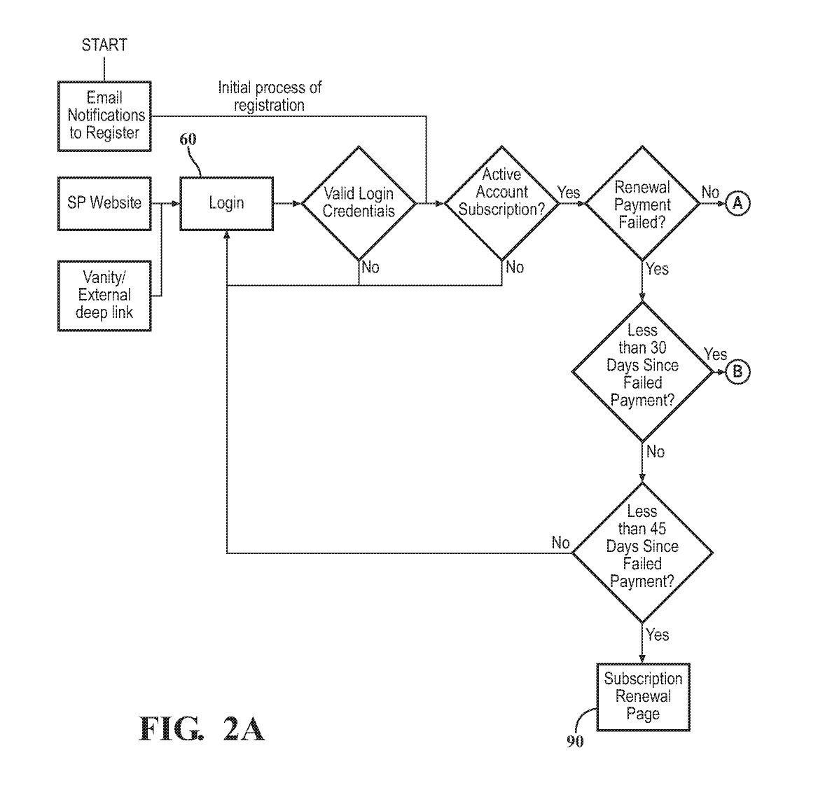

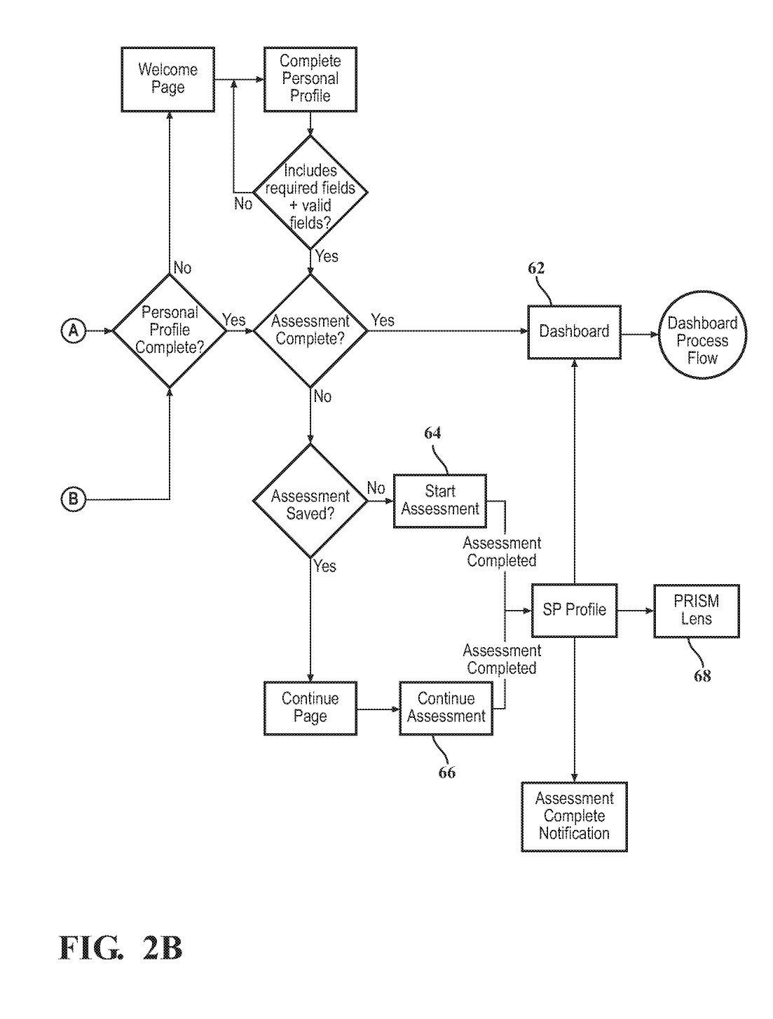

A business administration portion of the apparatus and method is shown in FIGS. 2A-11I and is accessible by at least one or more individual processors, such as a smartphone 32 or a computer 34 at a business site through the worldwide web 30 to the central computer processor system shown in FIG. 1.

As shown in FIGS. 2A and 2B, a business administrator can log-in in step 60 to open or access an assessment account. Such accounts include business profiles, subscription renewal services, etc. A dashboard 62 displayed on a computer monitor at the business site allows access by the business administrator to various parts of the employee assessment apparatus and method, including starting an assessment in step 64, continuing an assessment 66, and using the assessment results to create an employee profile in step 68.

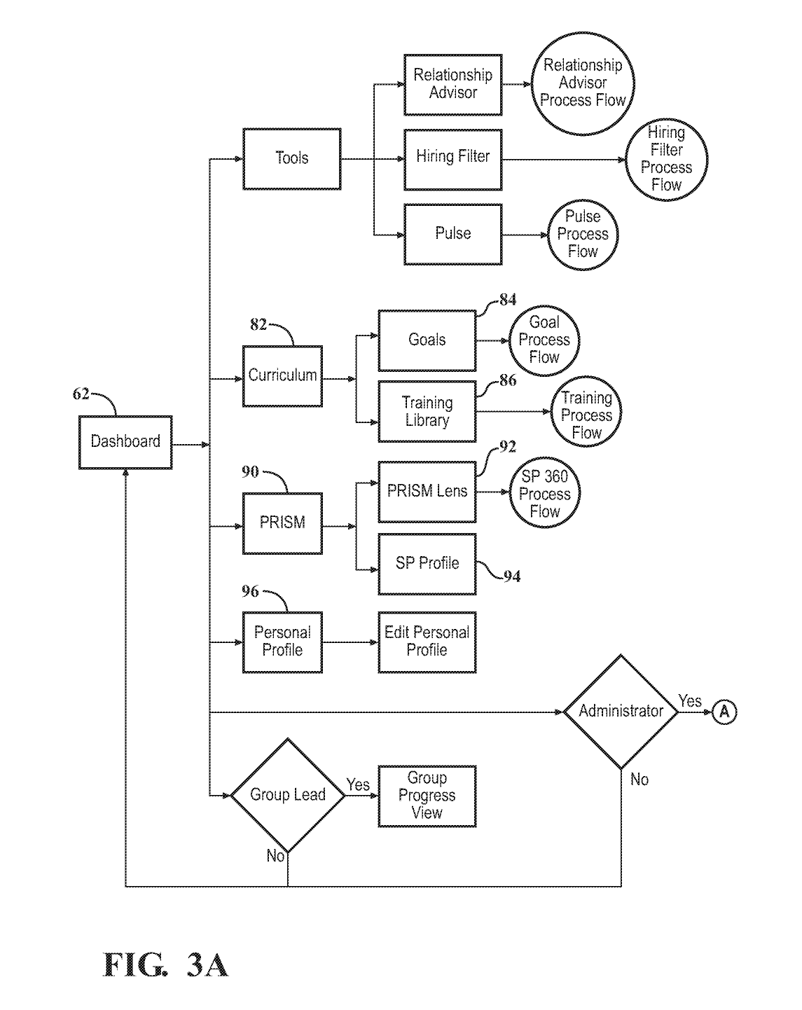

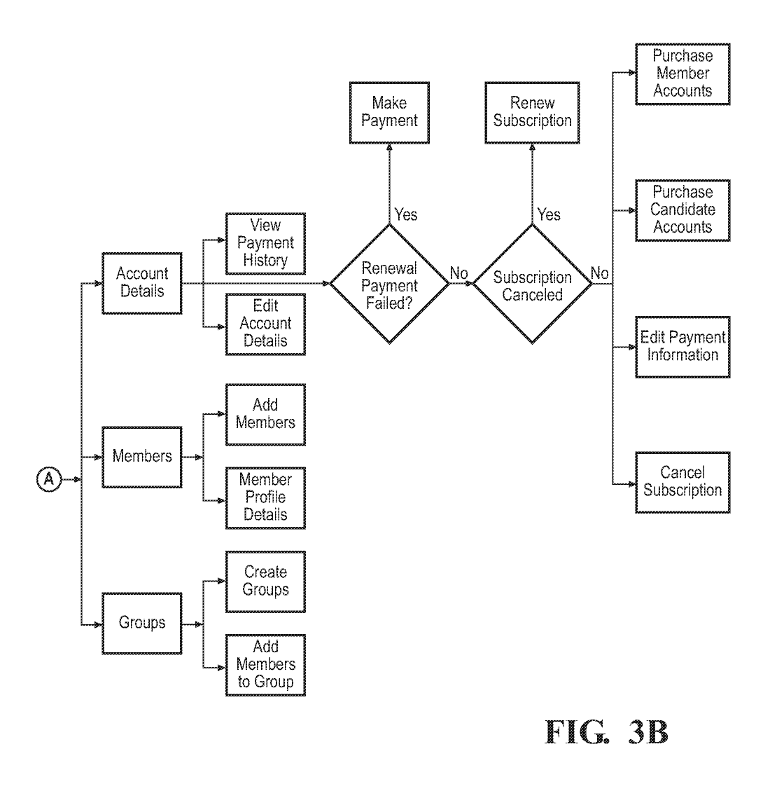

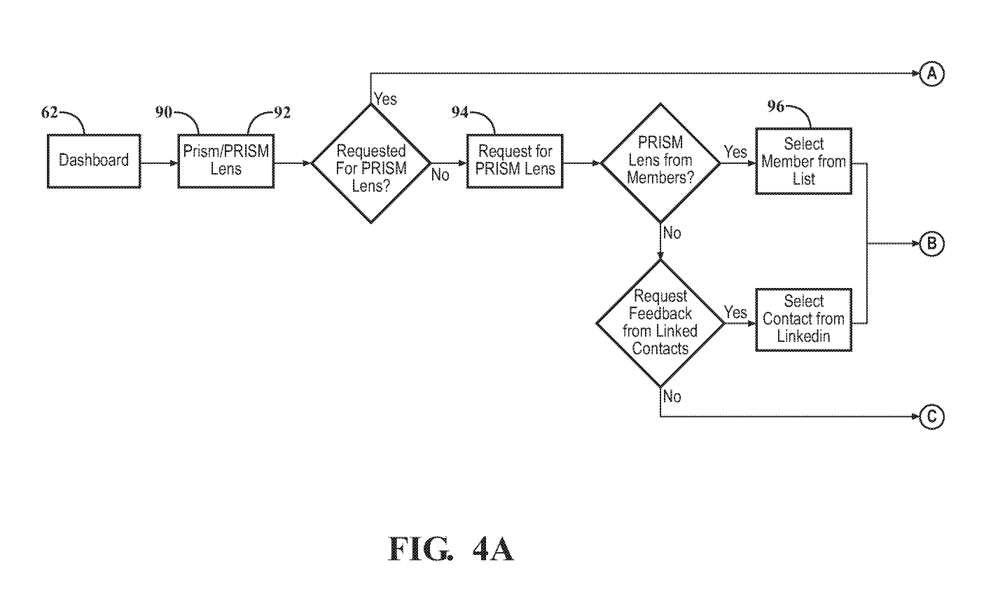

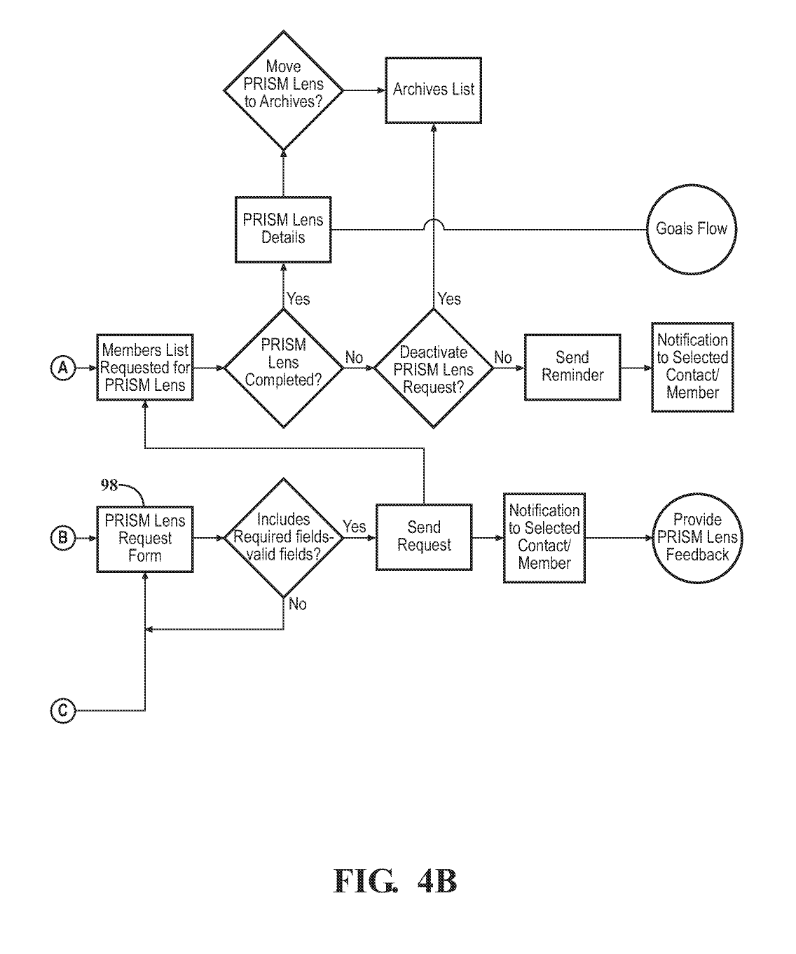

The dashboard 62, shown in FIGS. 3A and 3B, also allows access to various assessment or module features, such as a knowledge center 86. The dashboard 62 allows selection of a PRISM LENS™ module 92 allowing access to assessments 92 by an individual's peers and/or manager, or other individuals see FIG. 4, as well as to employee profiles 94. The dashboard 62 also allows access to a personal profile module 96 allowing the business administrator to edit the personal profile of any employee.

The dashboard 62 also provides additional versatility for the business to make payments as well as for the individual selected by the business or an outside individual to take an assessment 90. As shown in FIGS. 3A and 3B, a business administrator may assess account details 87 and make payments 89 in a number of different ways, such as all at once, periodic, automatically renewable, etc.

The business administrator may also authorize an individual selected by the business administrator to take an assessment test 90 to select, either at the businesses' or the individual's preference, to take all seven assessments of the PRISM™ assessment test 90 at one time, one at a time, in any partial sequence of less than the seven assessment, as well as providing a date deadline for completing all of the of the PRISM™ 90 assessment. In some instances, a business may not require all seven module of the PRISM™ 90 assessment described hereafter, in which case the business may direct an individual to take only selected assessments which are than presented to the individual. In the latter case, the individual may still have the preference to take all or some of the selected assessments at over a one-time preset time period etc.

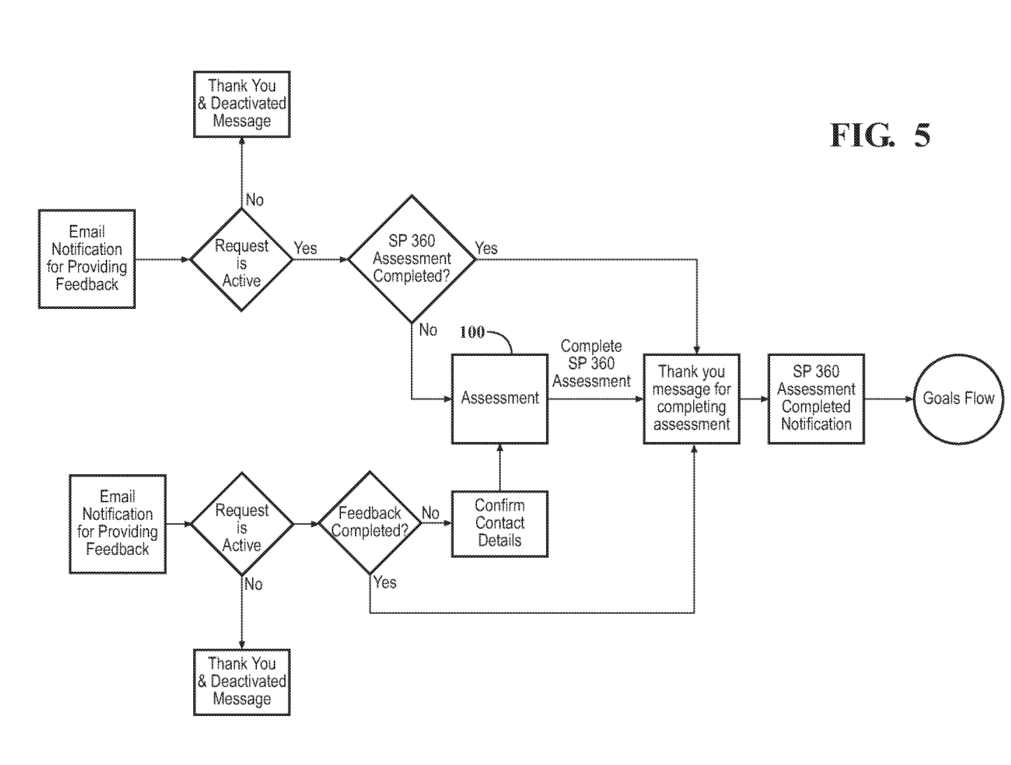

In the business mode, shown in FIGS. 4A, 4B, and 5, an administrator and/or employee may submit a request for an individual, such as an employee or non-employee, to take a PRISM LENS™ assessment 92 on another individual.

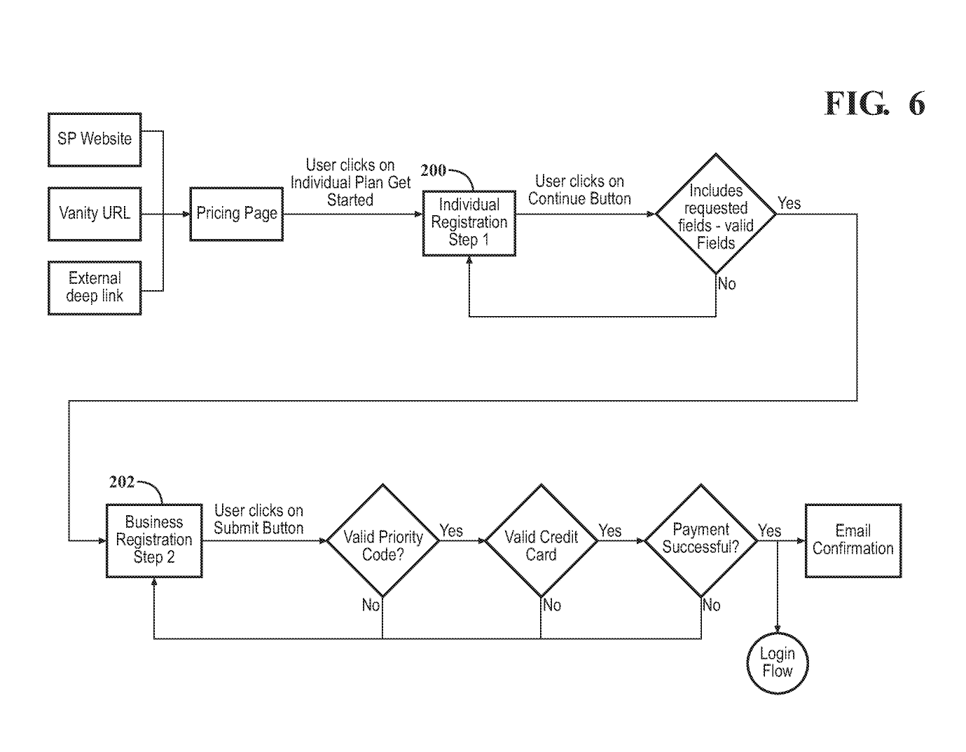

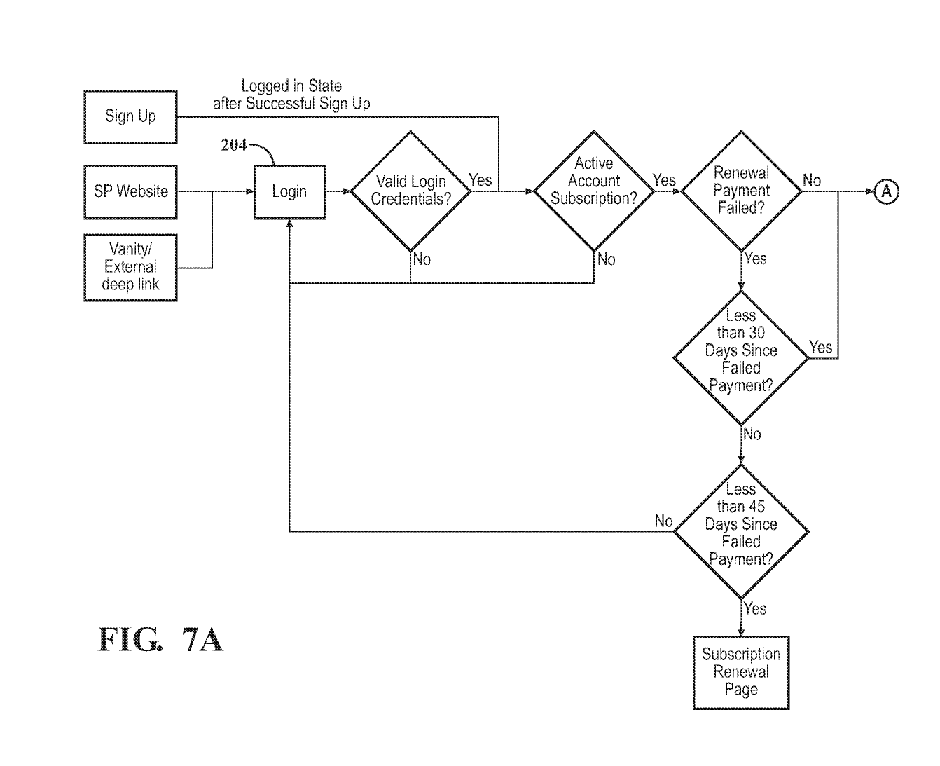

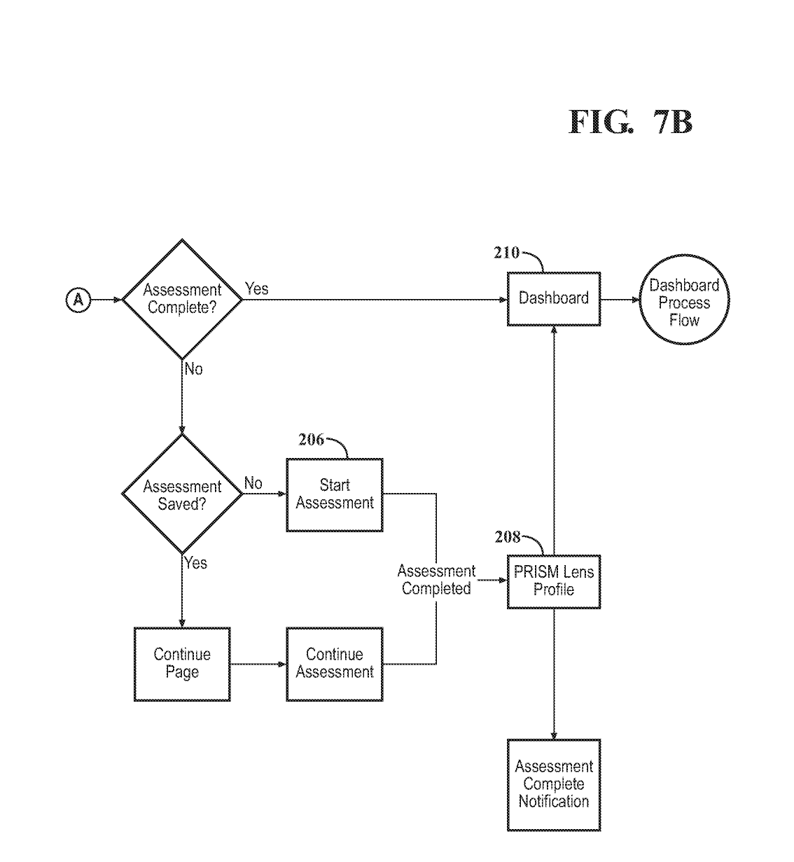

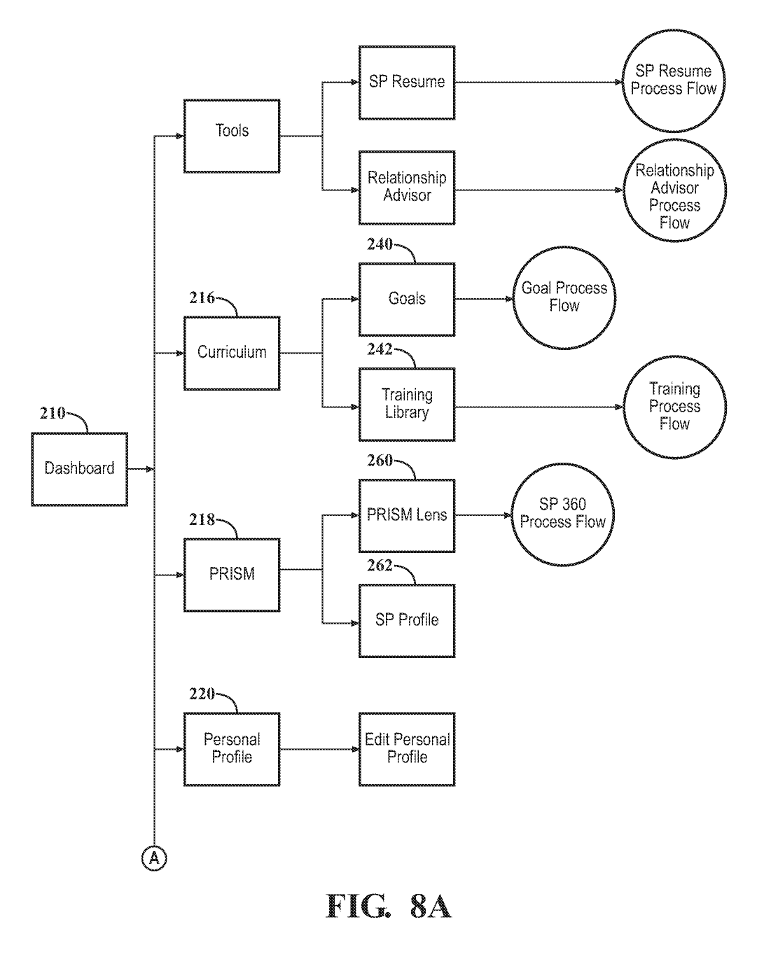

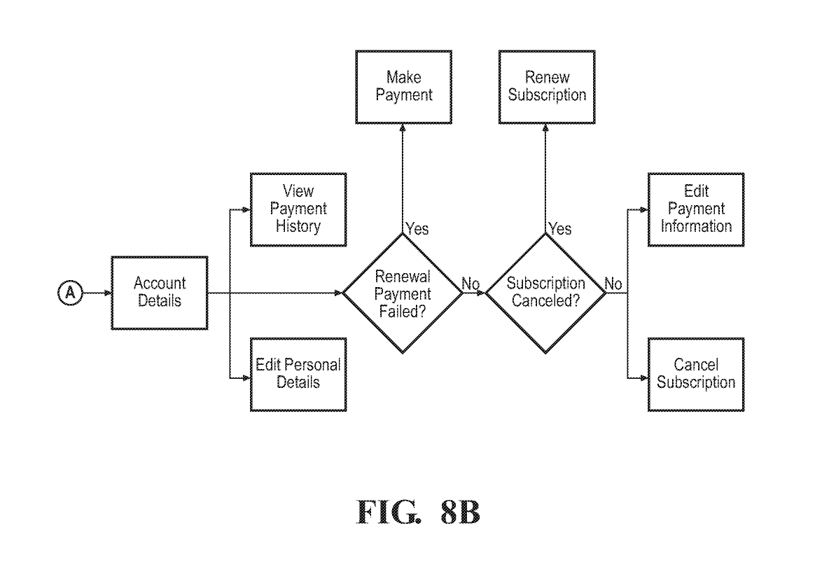

An individual may access the apparatus and method as described hereafter and shown in FIGS. 6-11I. Such individuals may be employees of a particular business who is registered with the apparatus and method, as described above, or totally unrelated to a particular business. An individual, as shown in FIG. 6, can individually register in step 200 or register through a business in step 202. After logging in in step 204 in FIG. 7A, an individual can start and/or continue the assessment 206 to create a 360 degree profile 208 which is input to a dashboard 210 for each individual in FIG. 7B. The dashboard 210 as shown in FIGS. 8A and 8B, allows access to a tools module 212, and a prism module 218.

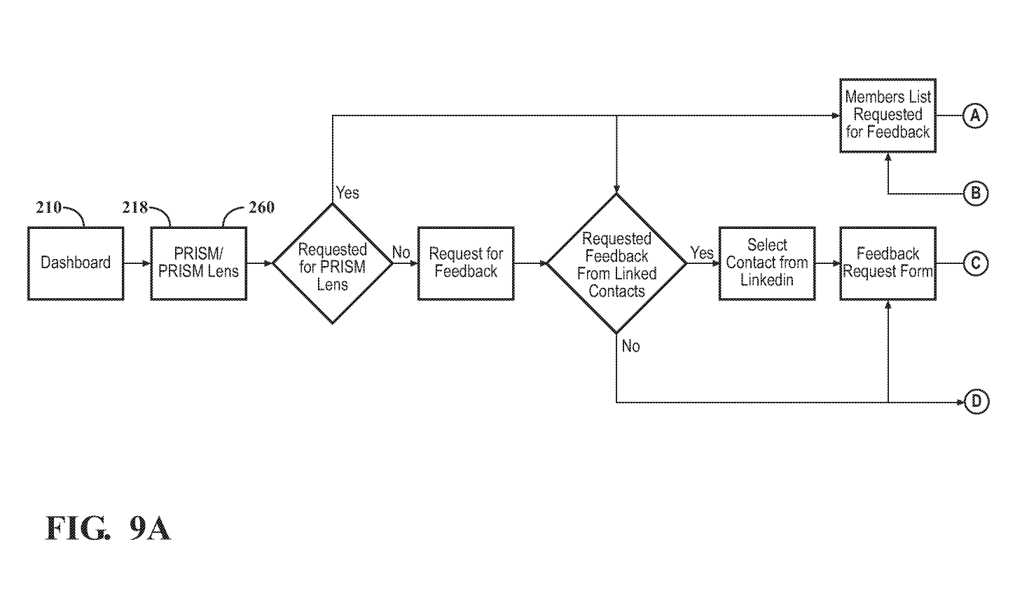

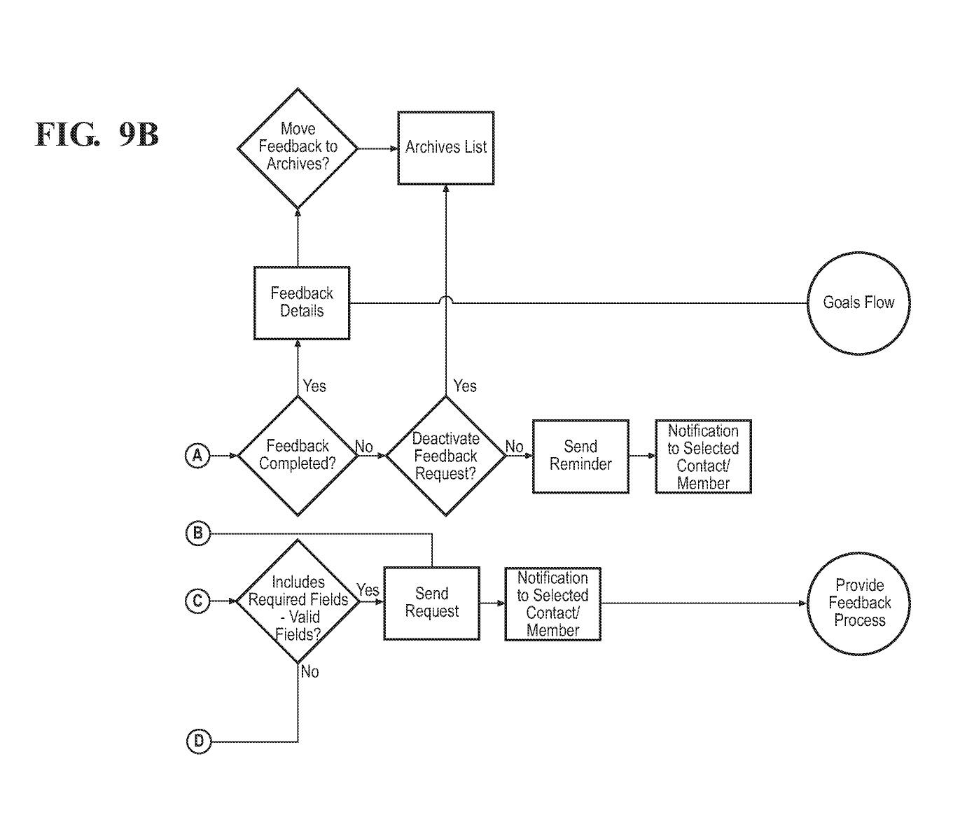

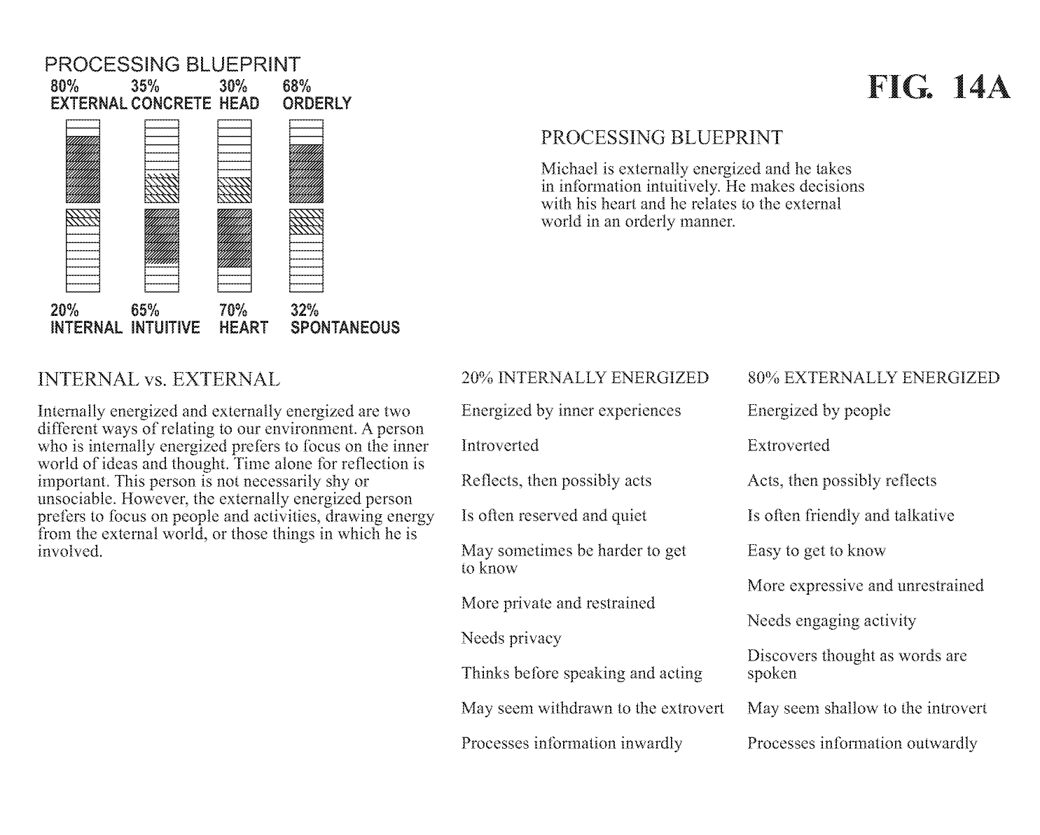

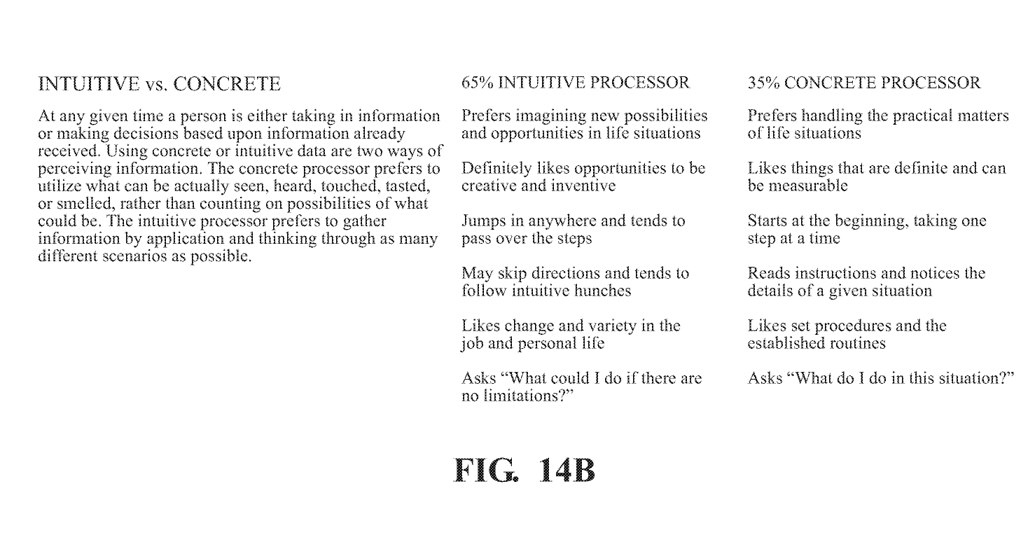

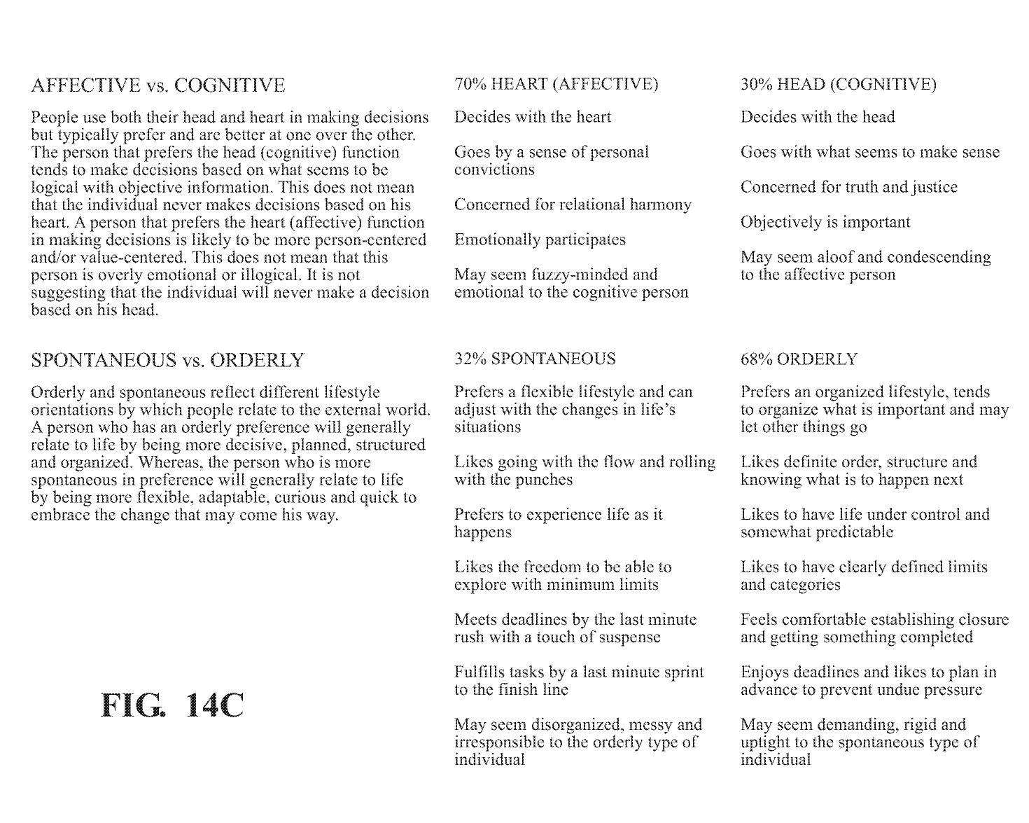

As shown in FIGS. 9A and 9B, an individual, through the PRISM LENS™ module 218, can access the PRISM LENS™ assessment 260 or review the individual's profile 262. As shown in detail in FIGS. 5, 8A, 8B, 9A, and 9B, when the individual selects the PRISM LENS™ assessment 260, the individual can select contacts or members for assessment by the sequence shown in FIGS. 14A-14C and receive feedback and actionable recommendations to improvements on the employees.

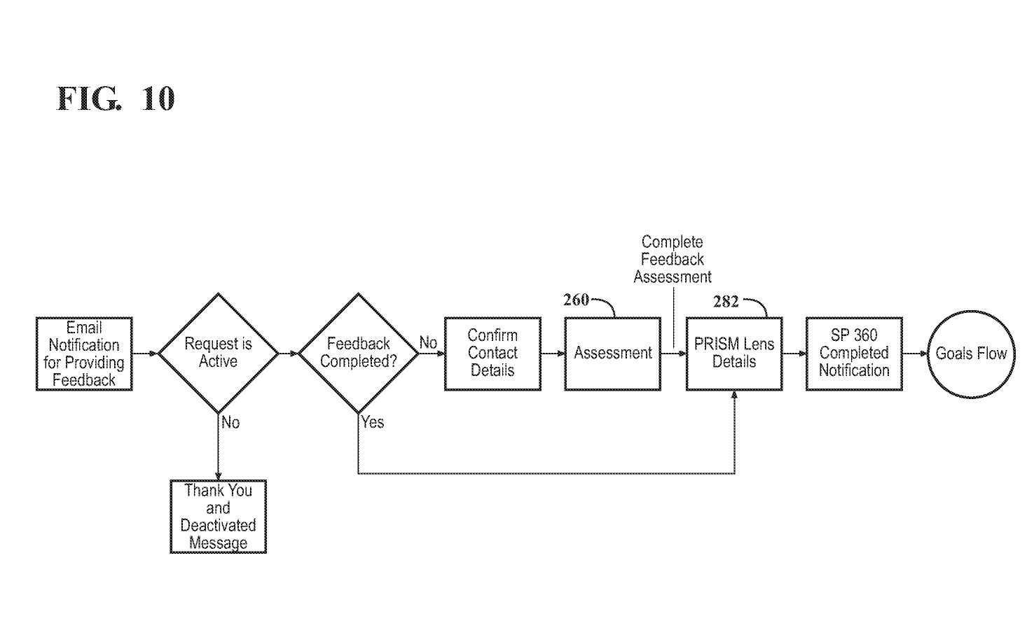

Finally, as shown in FIG. 10, an individual can access and complete the assessment 260 and receive an assessment report 282.

The PRISM LENS™ module 92 is further shown in detail in FIGS. 4A and 4B, when the PRISM™ module 90 and the PRISM LENS™ assessment tool module 92 are accessed through the dashboard 62. The PRISM LENS™ assessment module 92 allows a business administrator to select one or more individuals or employees of the business to provide an assessment of one employee. After receiving a request for the PRISM LENS™ assessment 92 in step 94, the business administrator can select one or more members from a list 96 and then send a request form 98 to the selected members.

The PRISM™ 90 assessment takes large amounts of data and converts the data to make it easy for businesses and employees to see, understand and put into action. The PRISM™ 90 assessment functions to identify multiple attributes including personality profile (primary and under pressure), processing blueprint, motivation, conflict management, fundamental needs, decision making and learning style.

The assessment apparatus and method creates a number of distinct primary employee personality profiles and employee personality profiles under pressure to assist a business administrator in determining whether or not to hire an employee, as well as determine how the employee is interacting with other business or business team members and the fit between employees on a team. Such personality profiles can include, for example:

Scientist Researcher

Navigator

Visionary

Architect

Catalyst

Tough & Tender

Connector

Instructor

Ally

Pragmatist

Strategist

Influencer

Peacemaker

Pioneer

Ambassador

The apparatus and method establishes characteristics for each of the personality profiles describing how an employee having a particular personality thinks, makes decisions, handles tasks and other personal relationships, work environment preferences. Each personality also has a list of work style characteristics, as well as how this particular individual with a specific personality may fit into a particular business team, how the individual is likely to communicate with team members, how the individual is influenced by negative perceptions of or by other team members. Such characteristics also enable a supervisor to best supervise and encourage an individual with a particular personality profiles.

These personality profiles enable a business administrator, when forming a team of employees or hiring a new employee as an addition to an existing team, to best choose the team members or the new employee to fit into the team, and to meet the goals of the team.

These personality types and personality under pressure types are established by the assessment program which asks an individual a series of questions describing workplace scenarios, preferences, motivation, work styles, etc. Based on the answers, the assessment program of the apparatus and method establishes a particular personality type for an individual. The established characteristics associated with that individual's personality can then be established for the business administrator.

This assessment information is valuable to the business administrator when forming a team, determining the efficiency of an established team, or when conferring to hire a new employee and placing that employee in a particular team. This enables the business administrator to achieve the business goals in an efficient manner, as well as advancing the growth of each employee.

A business employee, as well as non-business individuals, who take the PRISM™ assessment, are provided with a custom portrait of their personality, how they operate in a business environment when making decisions, interacting with others, achieving goals, for advancing the individual to meet business-related and/or life goals.

Appendix A depicts a number of different personality profiles used by the present assessment apparatus and method. These personality profiles are examples only as the number of personality profiles used by the apparatus and method can include less than or more than the different personality profiles described in Appendix A.

The personality profiles shown in Appendix A as well as the actual assessment as described hereafter are provided by way of an example as any available assessment program may be employed in the present apparatus and method. The personality characteristics of any individual from any available assessment can be correlated to the sixteen personality profiles and the seven assessments described hereafter.

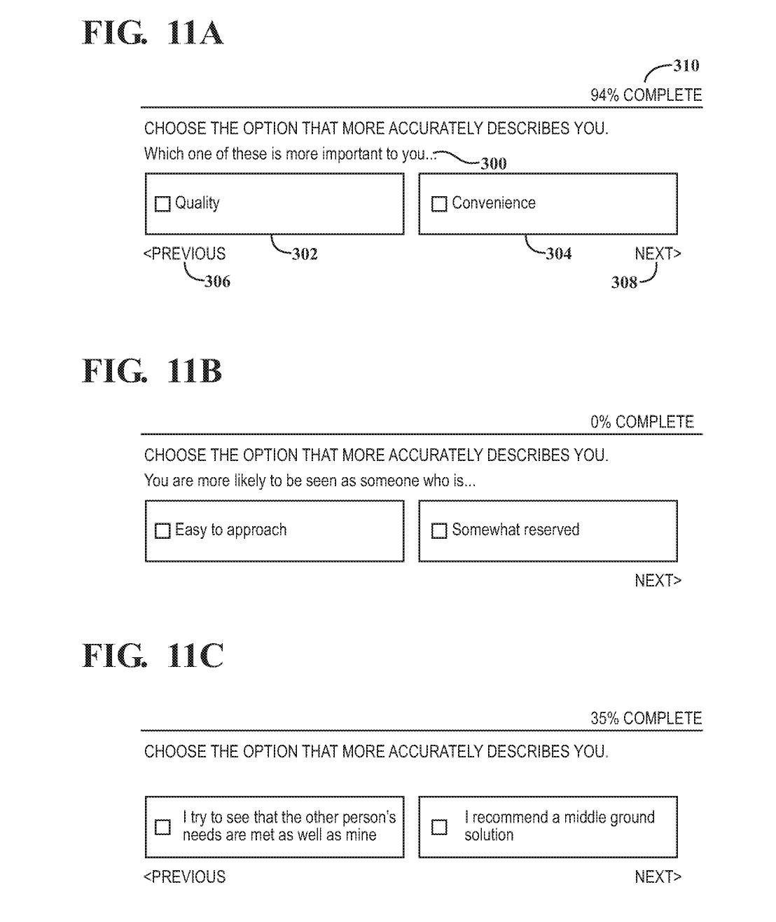

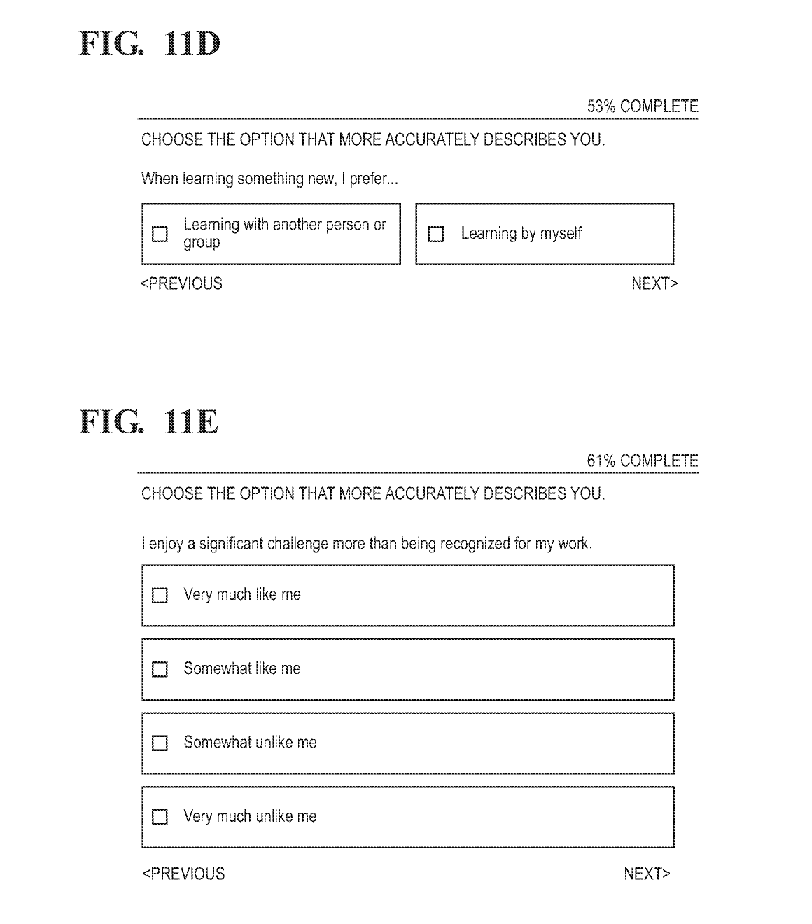

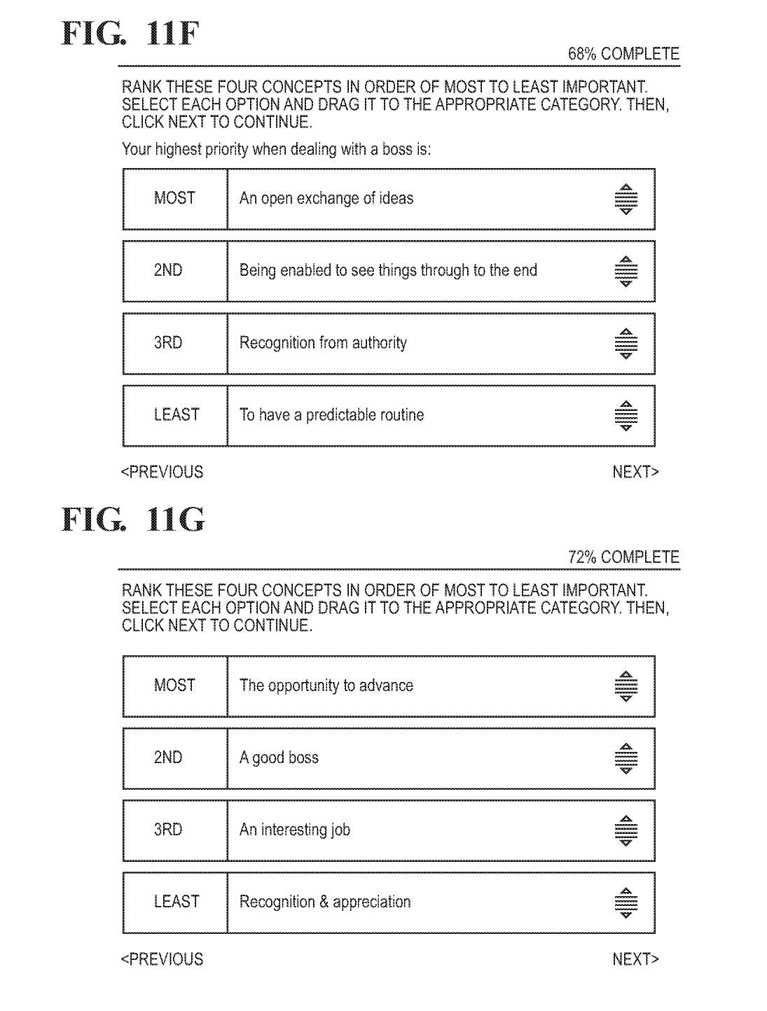

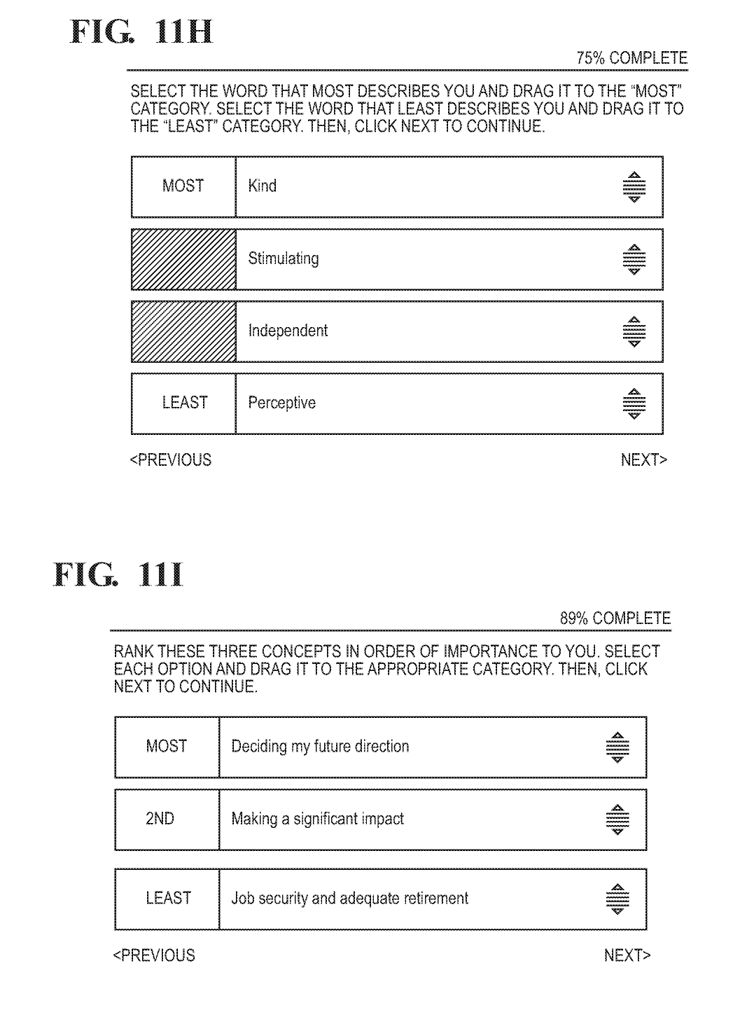

FIGS. 11A-11I depict different forms of assessment questions presented to the individual as part of the PRISM™ 90 assessment. The questions are displayed on a monitor and generally ask an individual to choose the characterization that most generally describes that individual's view of the assessment question. The page displayed on the monitor also enable the individual to easily return to a previous question or to advance to the next question. The display also depicts the percent completion 310 of the entire assessment 90.

The data driven assessment apparatus and method presents the personality and the individual characteristics in easy to read scales, charts and graphs to enable a business administrator or the individual to easily ascertain the individual's personality, decision making tendencies motivation, etc.

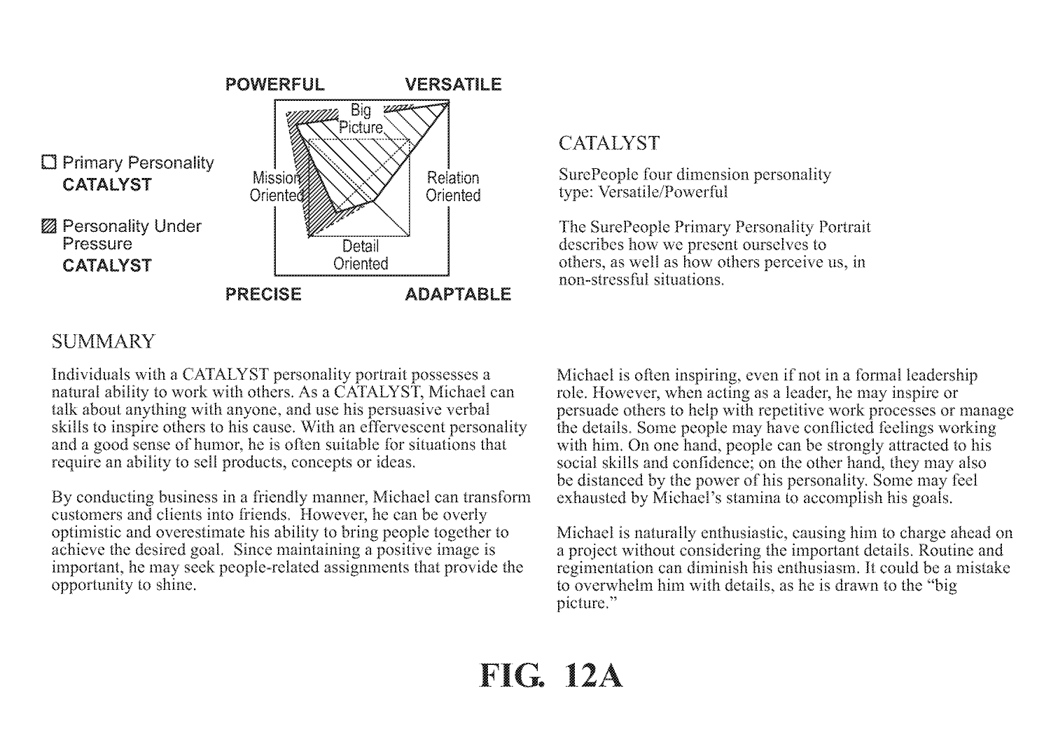

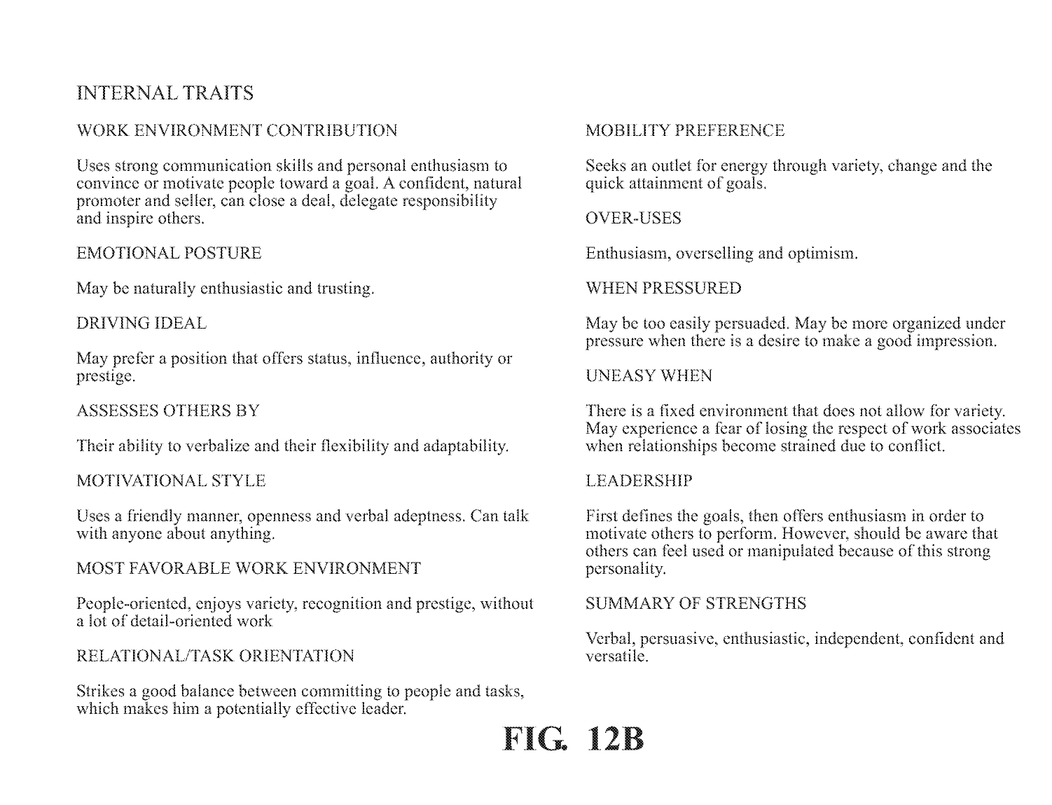

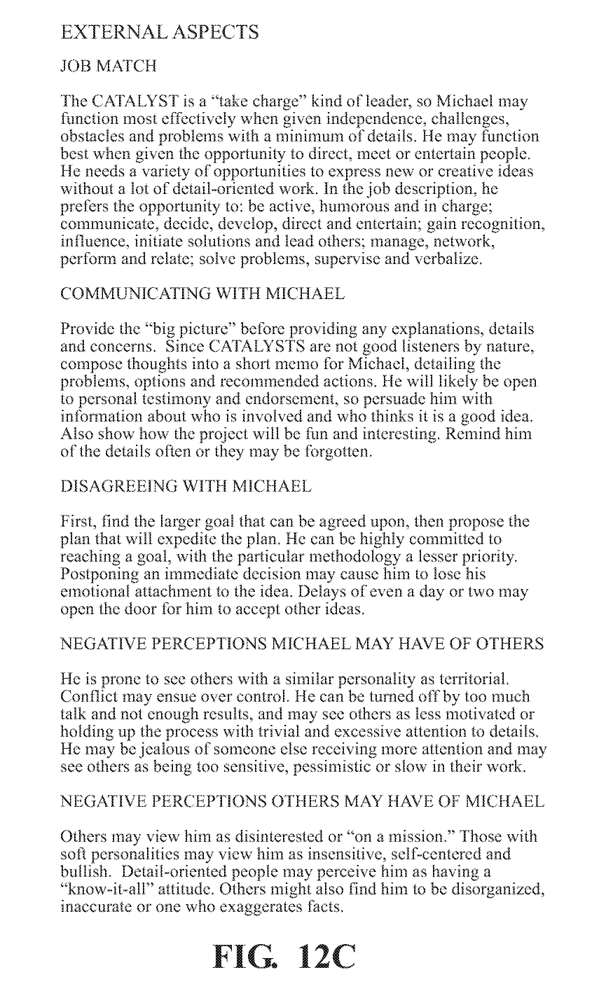

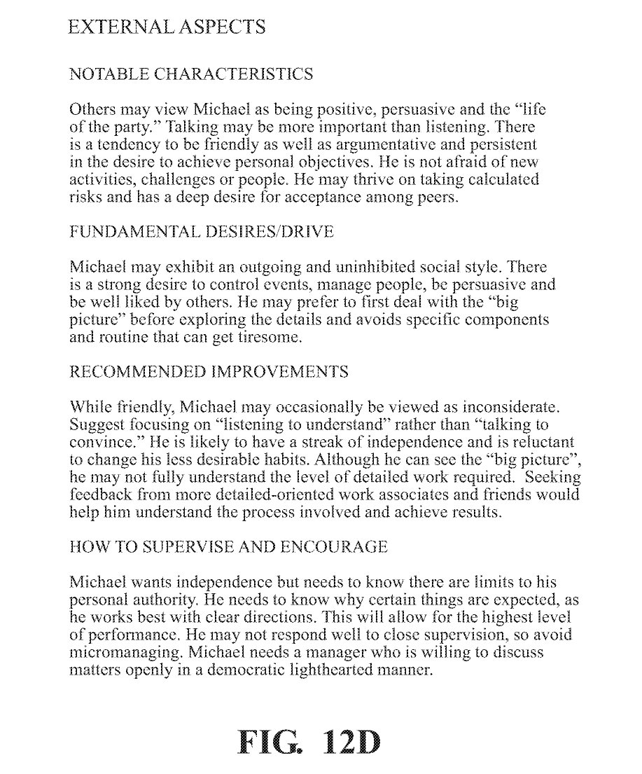

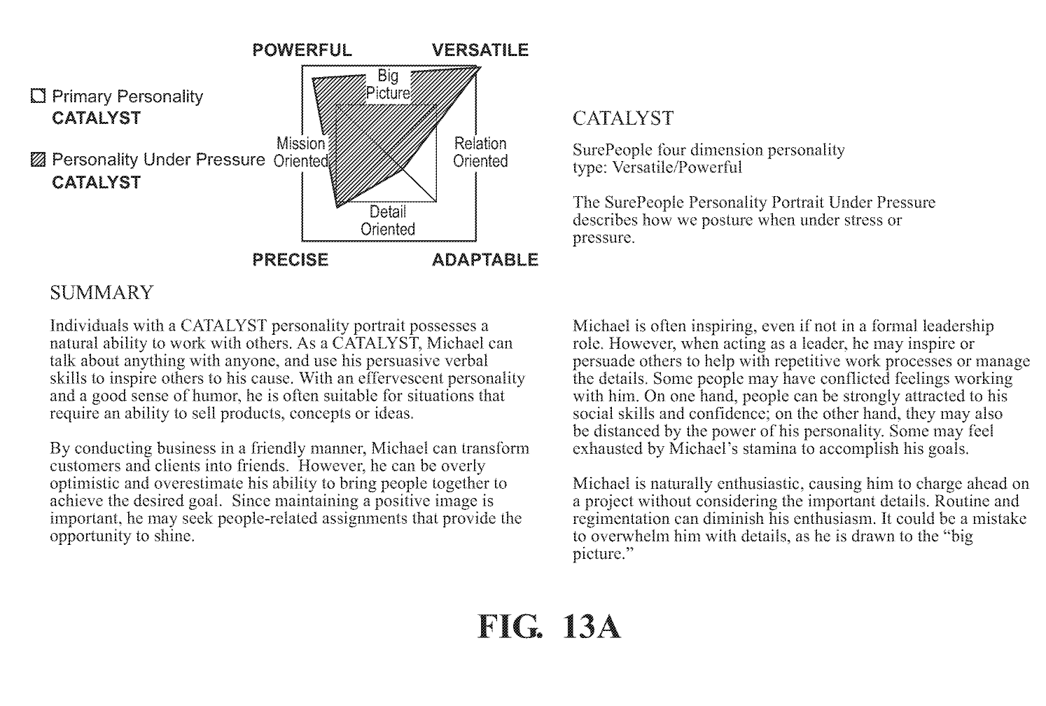

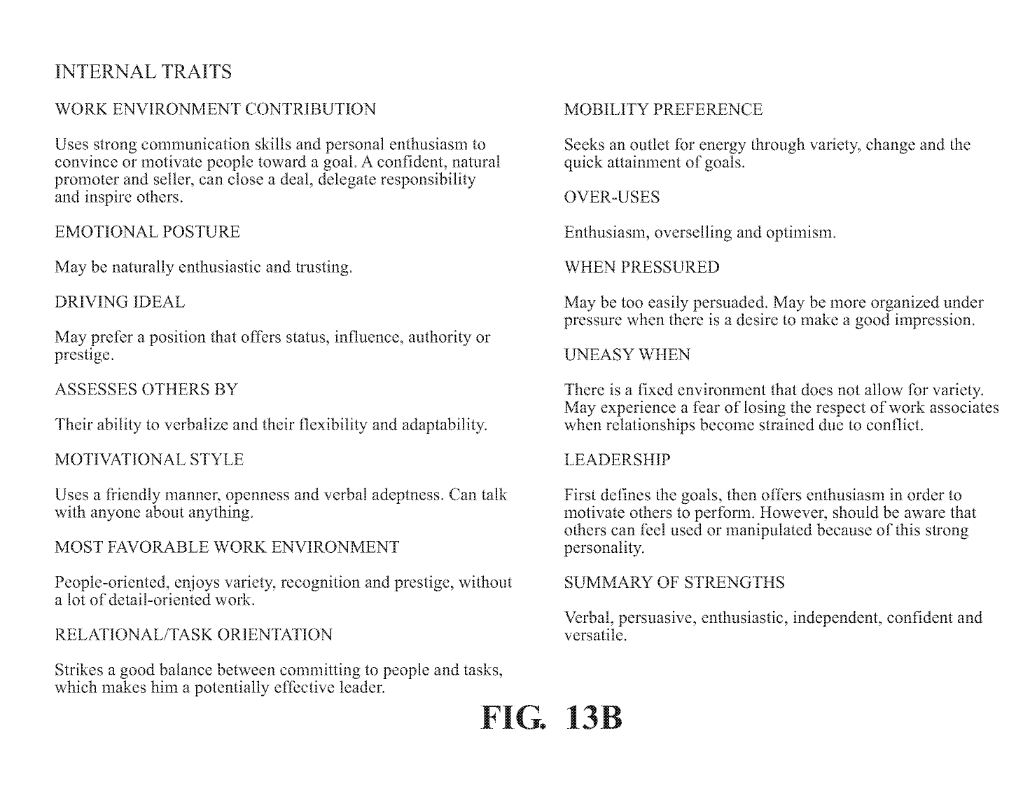

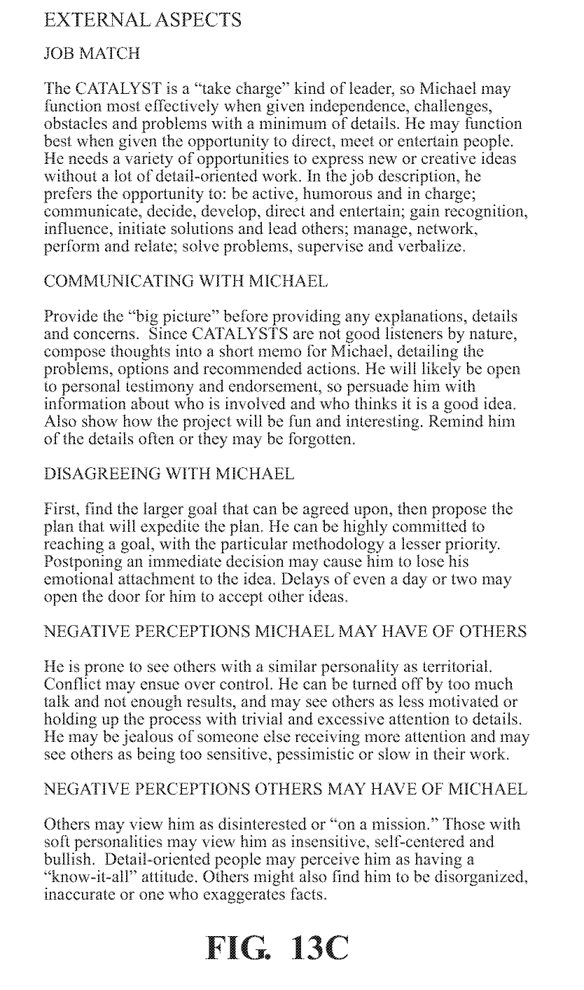

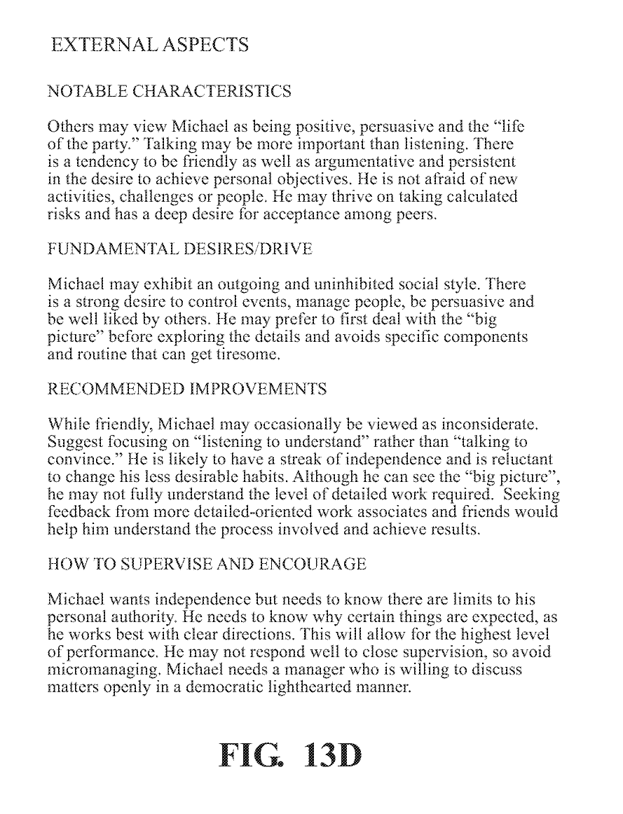

The PRISM™ 90 assessment reveals both primary personality in FIGS. 12A-12D and personality under pressure in FIGS. 13A-13D within a four dimensional scale that measures power, versatility, precision and adaptability. The PRISM™ 90 assessment delivers a comprehensive summary filled with insights and a unique classification for people to see, read, and understand their personality.

Not everyone absorbs information in the same way. After processing information, some people will make decisions more with their head, while others tend to take action with their heart. The processing blueprint feature of PRISM™ 90 assessment shown in FIGS. 14A-14C shows how employees approach people, activities, thoughts, and causes in their lives based on four distinct scales, including:

Internal vs External: How people are energized by outside factors or inner thoughts and concepts.

Intuitive vs Concrete: The way people take in and perceive information.

Head vs Heart: If people tend to make decisions guided more by their thoughts or feelings.

Spontaneous vs Orderly: How people prefer to approach and relate to live.

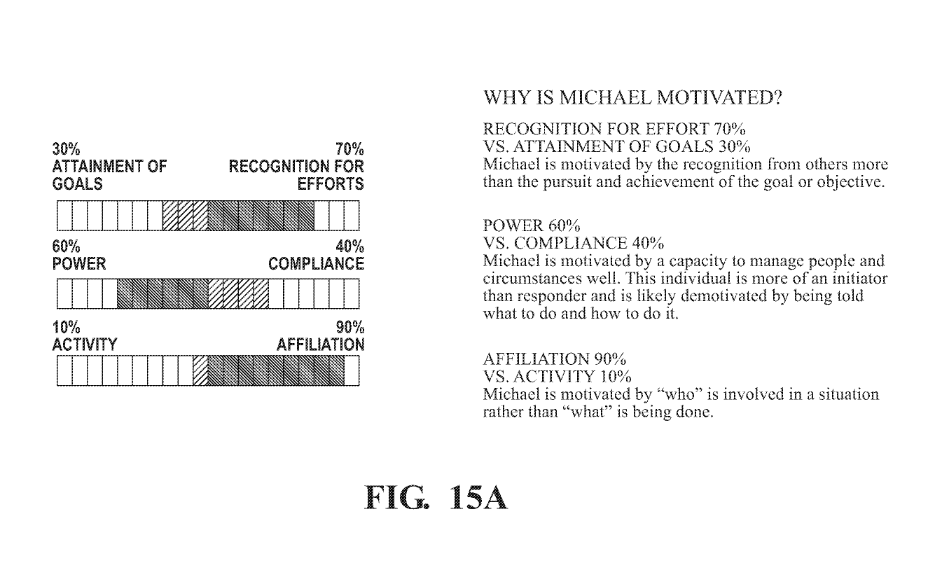

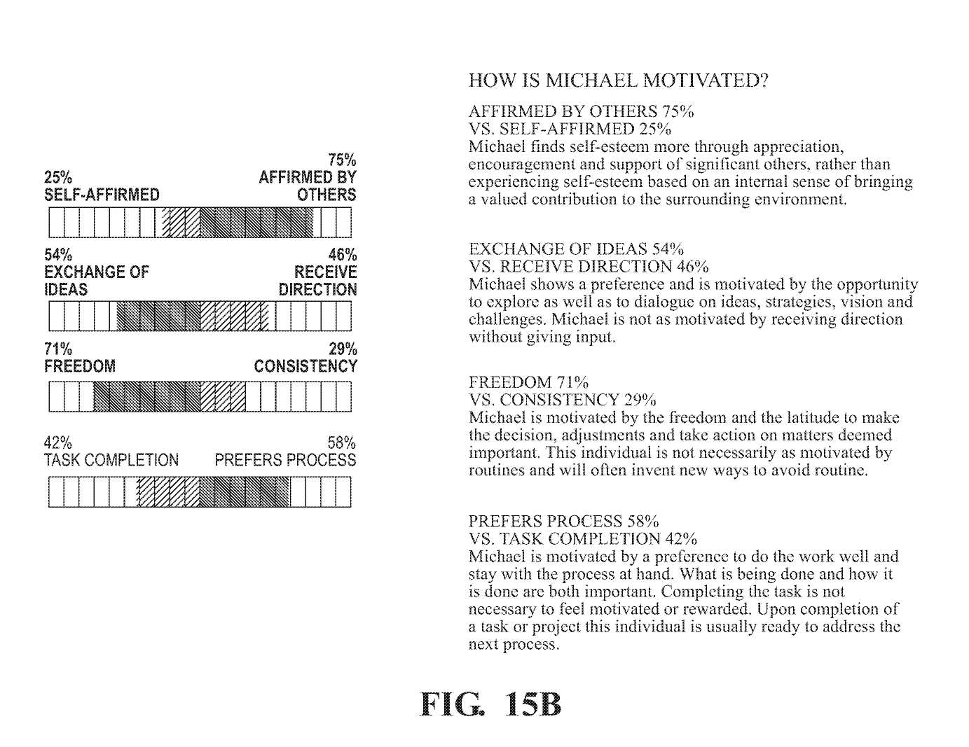

Motivation is also a determination of the PRISM™ 90 assessment, as shown in FIGS. 15A-15C. Some employees may want recognition while others need a little encouragement to keep moving forward. The motivation output of the PRISM™ 90 assessment uncovers how people are motivated, why they are motivated, and what they are motivated by.

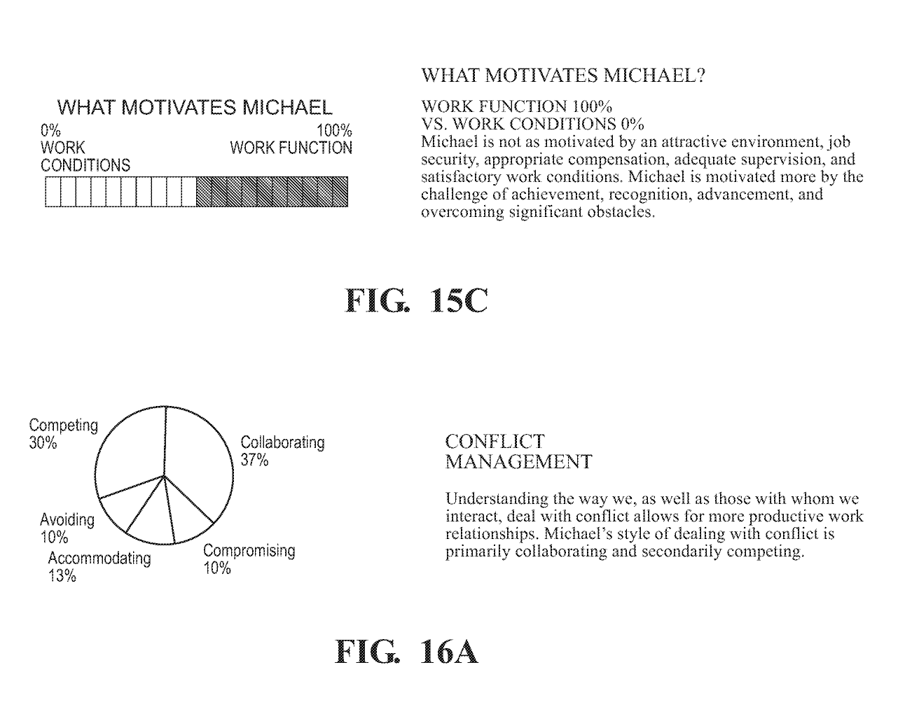

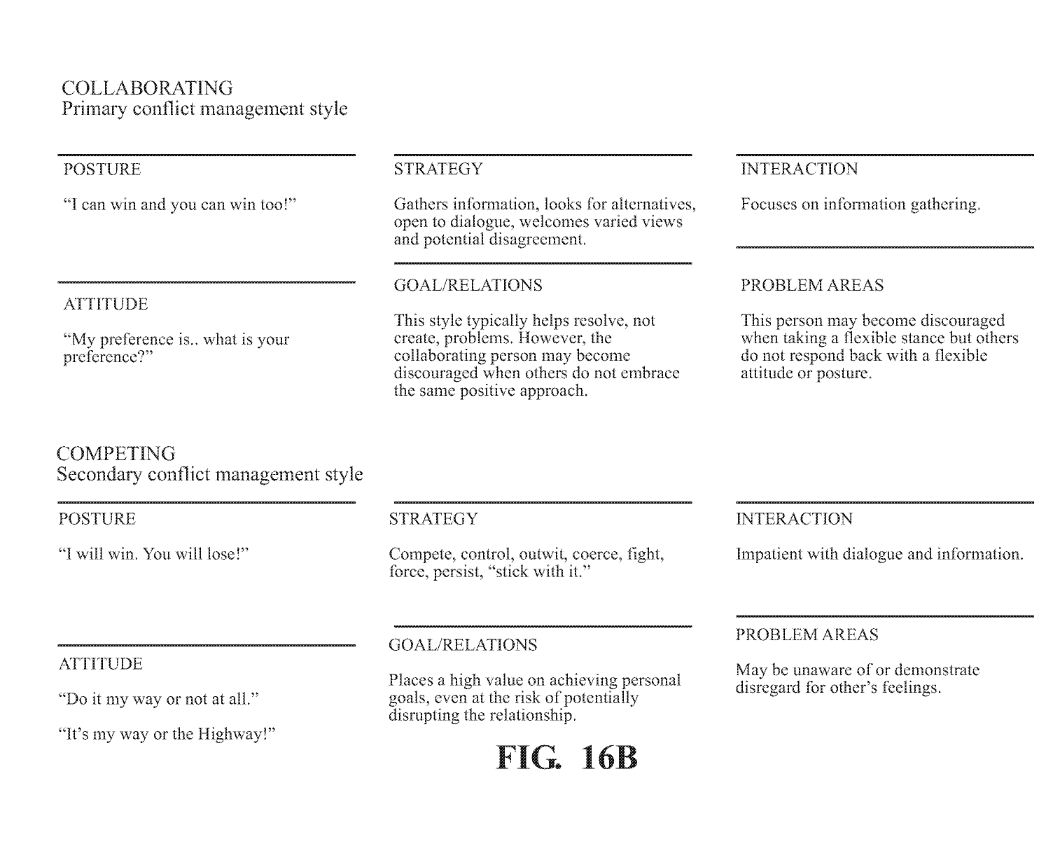

How employees deal with workplace conflicts is important since such conflicts arise all the time. The conflict management output of the PRISM™ 90 assessment, as shown in FIGS. 16A and 16B reveals how a business's employee's or prospective employees work through issues with others, by competing, calibrating, avoiding, accommodating or compromising. Such insights allow business administrators greater understanding on how the business's employees interact with one another and deal with conflict, thereby allowing the business administrator to foster more productive working relationship.

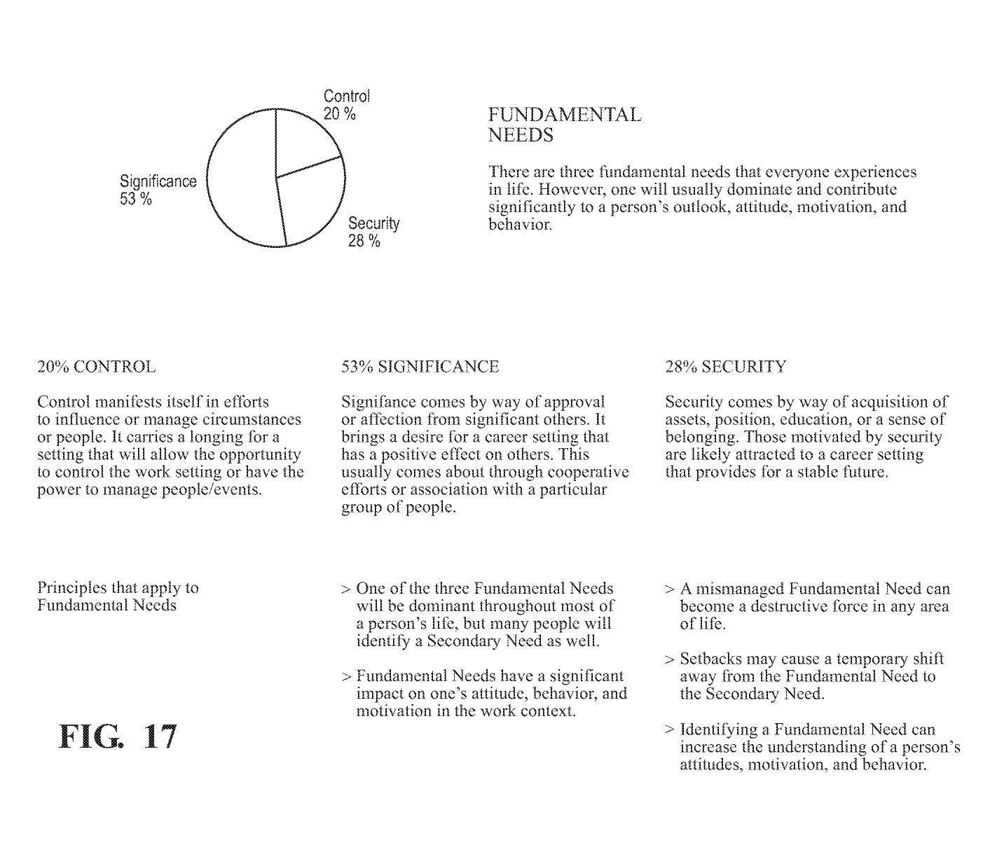

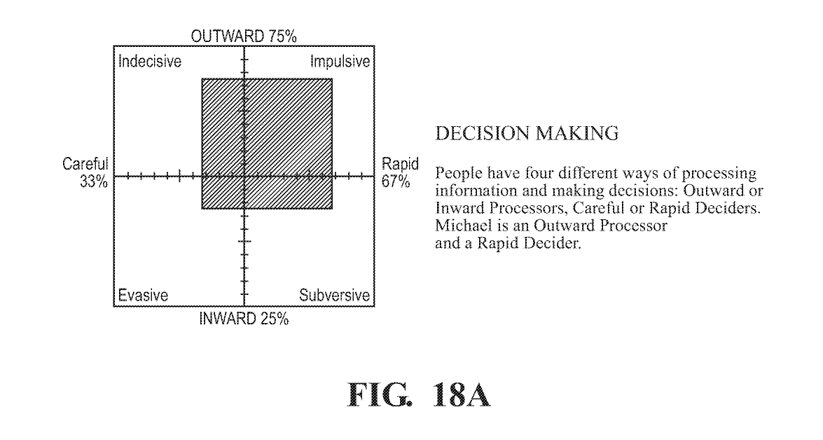

All employees have fundamental needs that drive the employees and contribute to their outlook, attitude, motivation, and behavior. The fundamental needs output of the PRISM™ 90 assessment, as shown in FIG. 17 measures a person's desire to maintain control, have security or achieve significance. These insights provide a business administrator with a deeper understanding about their employee's needs for helping team members to appreciate and communicate with each other.

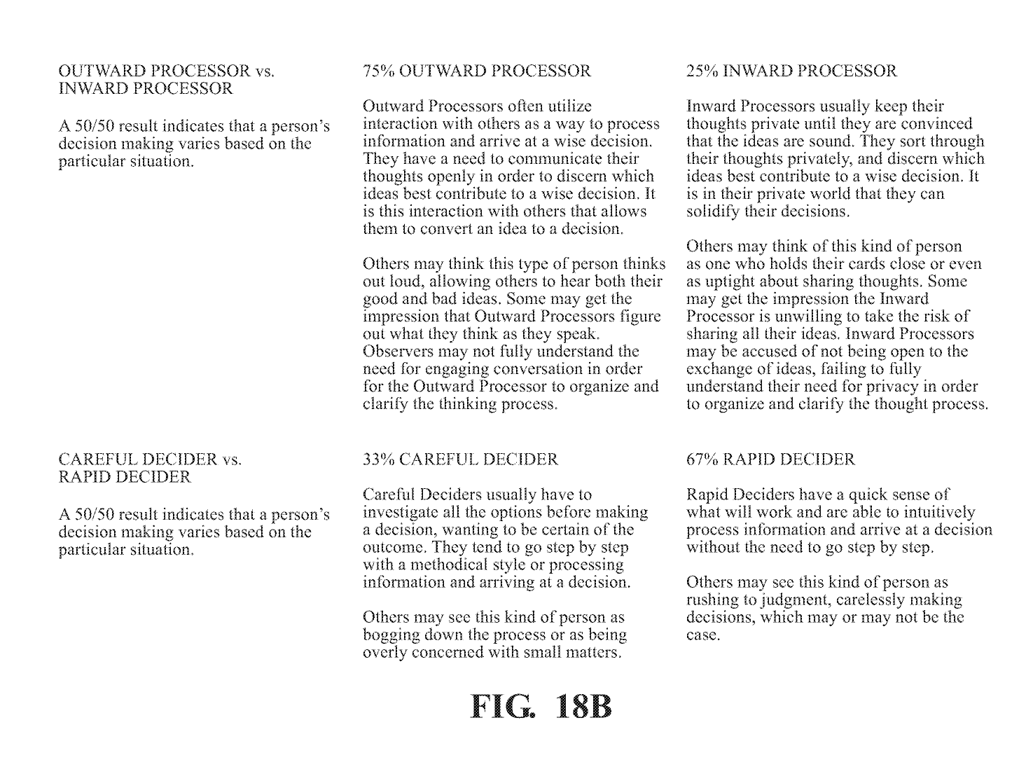

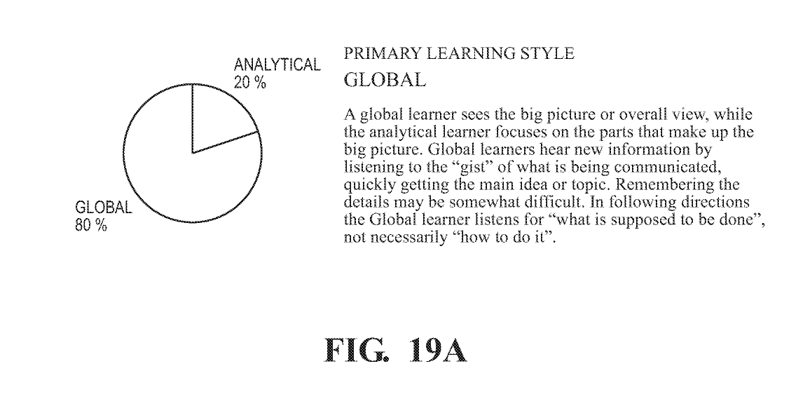

Every employee has their own unique style and internal or external rationale for making decisions. The decision making output of the PRISM™ 90 assessment, as shown in FIGS. 18A and 18B, measures this critical component of an individual based on four unique yet related decider scales, including outward, inward, careful and rapid.

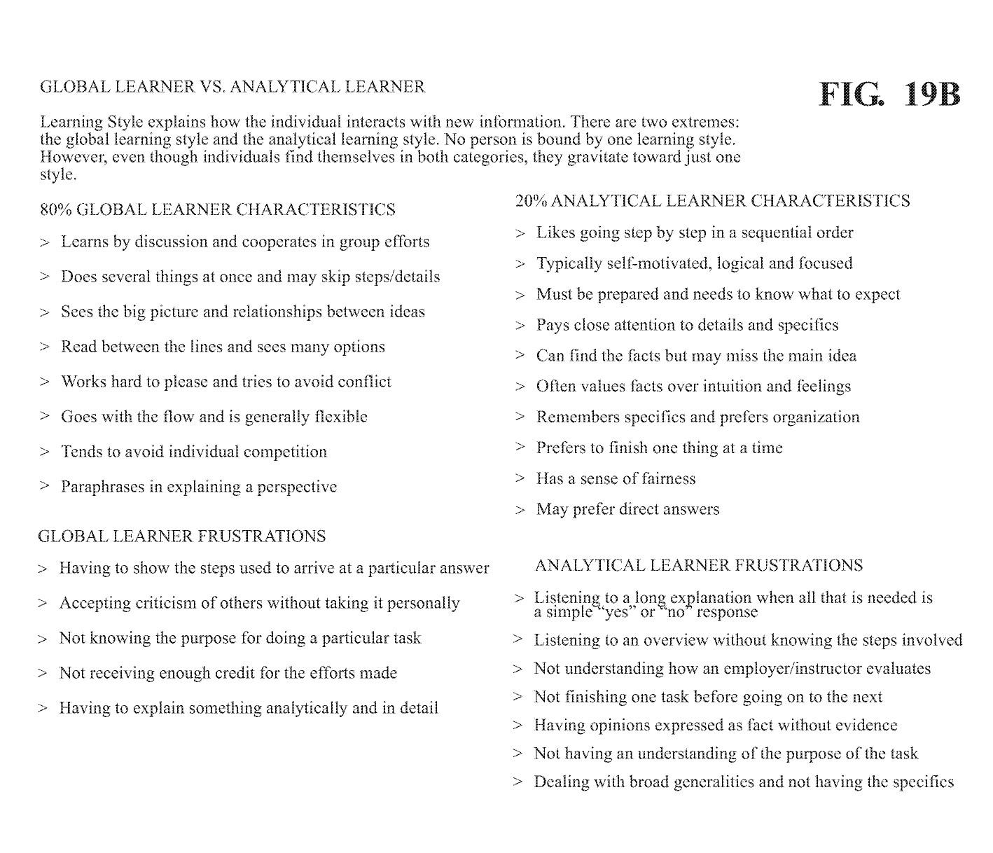

Understanding how people learn can assist a business and employees to communicate more effectively with one another in a team environment in another factor. The learning style output of the PRISM™ 90 assessment, FIGS. 19A and 19B, reveals how people process information and details with two distinct scales of learning—global and analytical. A global leader tends to focus on the big picture and might take more risks than others. An analytical leader pays close attention to the details and strives for accuracy.

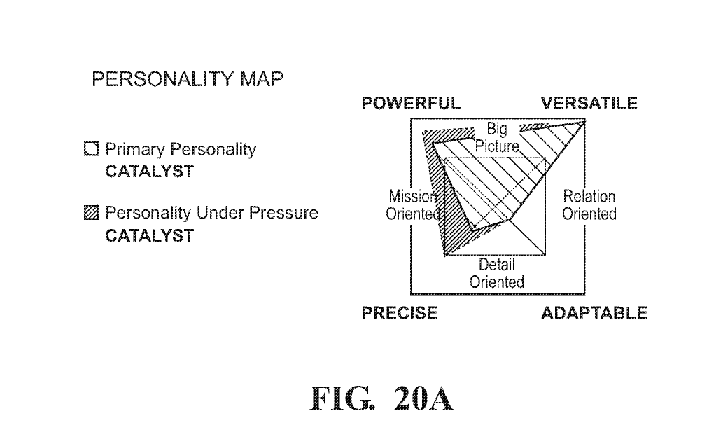

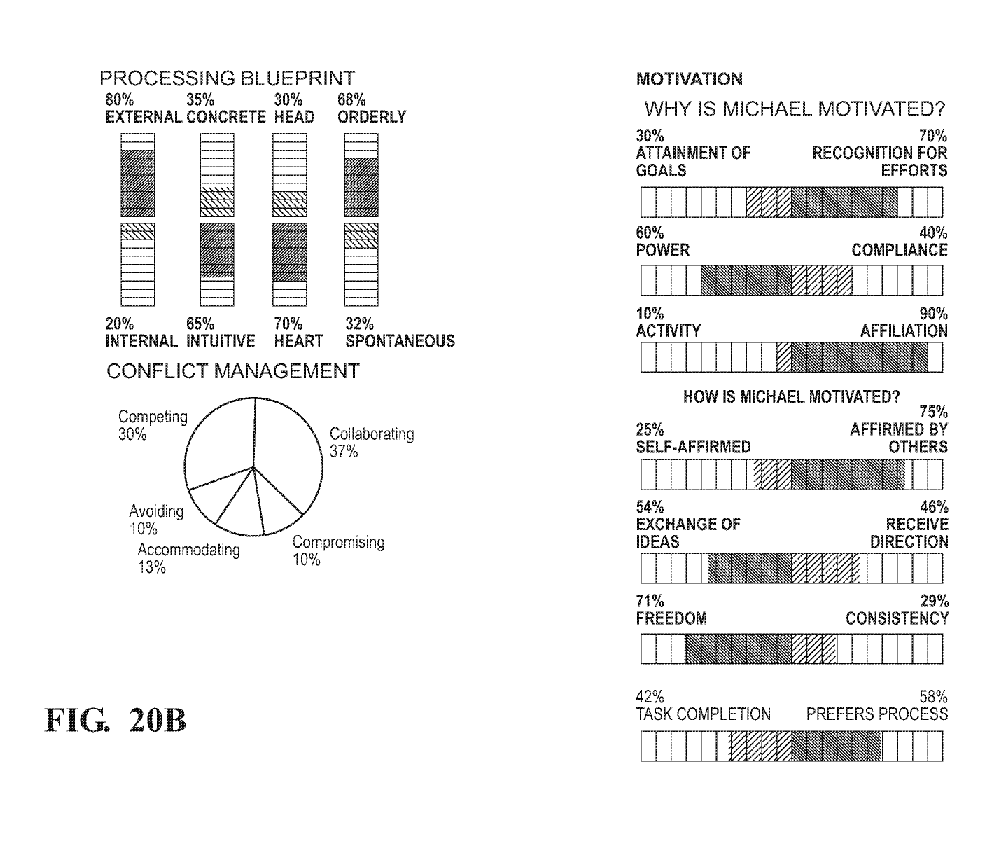

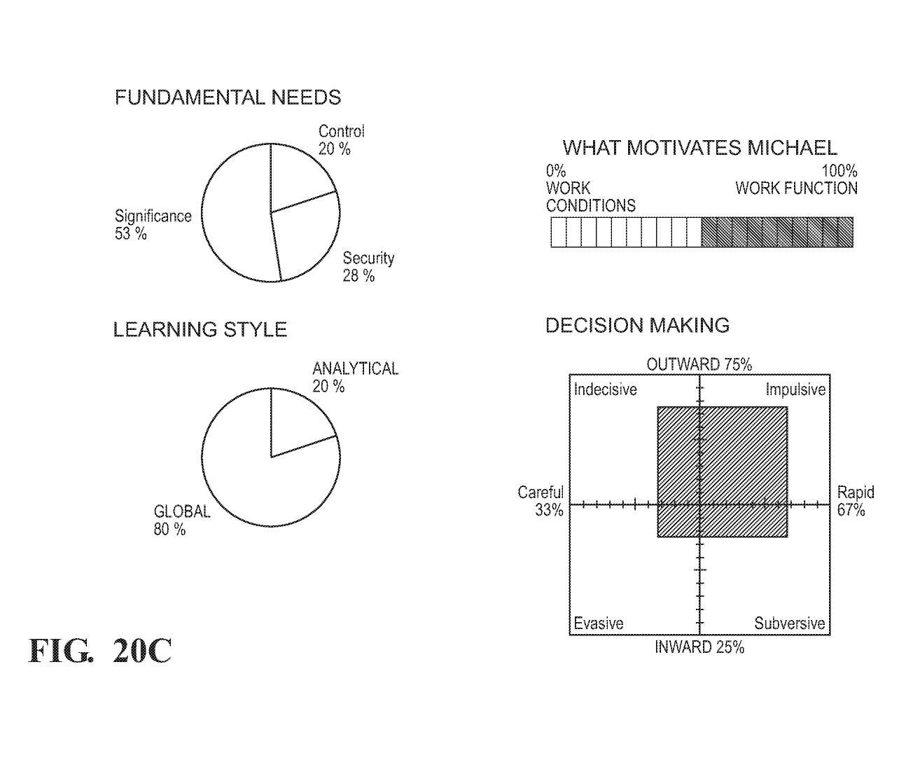

FIGS. 20A-20C all of the charts and graphs shown in FIGS. 11A-19B in a one-page summary for ease of use by a business administrator as well as the individual to determine the individuals' personality characteristics.

1. A method for generating an individual's personality assessment data, the method comprising:
accessing a memory by a computer processor coupled through a network to at least one individual processor;
administering an assessment through the at least one individual processor to determine an individual's assessment data;
analyzing the assessment data by the computer processor; and
generating the assessment to the at least one individual processor containing at least one assessment of the individual.

2. The method of claim 1 further comprising:
storing an assessment test in a medium accessible by the computer processor; and
the step of administering the assessment test includes administering the assessment test through the network to the at least one individual processor by a display.

3. The method of claim 2 wherein the step of administering an assessment comprises:
administering an assessment test to the individual in response to a third party request through an individual processor to the computer processor for the individual to take the assessment test.

4. The method of claim 2 comprising:
transmitting the assessment from the computer processor to at least one of the individual processor of the individual and an individual processor of a third party.

5. An apparatus for generating an individual's personality assessment data comprising:
a computer processor assessing a memory storing an assessment test;
at least one individual processor coupled through a network to the computer processor; and
a visual display coupled to the at least one individual processor for displaying an assessment test and an assessment report.

6. The method of claim 1 wherein the step of administering the assessment test includes:
administering assessments of primary personality, personality under pressure, processing blueprint, motivation, conflict management, fundamental needs, decision making and learning style.
